(19) United States
(12) Patent Application Publication
(10) Pub. No.: US 2010/0076255 A1
(43) Pub. Date: Mar. 25, 2010

(54) Method and apparatus for treating a prolapse related condition

(75) Inventors: Peter Robertson, New South Wales (AU); Anthony Clyde Neason Stephens, New South Wales (AU)

Correspondence Address: JONES DAY, 222 EAST 41ST ST, NEW YORK, NY, 10017, US

(73) Assignee: Continence Control Systems International Pty Ltd, Chastwood, NSW (AU)

(21) Appl. No.: 12/310,716

(22) PCT Filed: Sep. 12, 2007
(86) PCT No.: PCT/AU2007/001356

§ 371 Date: Mar. 5, 2009

(30) Foreign Application Priority Data
Sep. 12, 2006 (AU) 2006905028

(51) Int. Cl. A61N 1/36; A61F 2/04; A61N 1/05

(52) U.S. Cl. 600/30; 600/37; 607/41; 607/138

(57) ABSTRACT

The present invention provides a method and apparatus for treating a prolapse related condition. The apparatus includes an implantable stimulator arranged to be implanted within a patient. The stimulator provides a signal for stimulation of tissue in the pelvic region.

### FIELD OF THE INVENTION

The present invention relates to a method and apparatus for treating a vaginal prolapse related condition, and, more particularly, but not exclusively, to a method and apparatus for treating a condition associated with vaginal prolapse and/or associated with treatments for vaginal prolapse, such as surgery.

### BACKGROUND OF THE INVENTION

Failure or wear in the network of muscles, ligaments, fascia and skin in or around a woman's vagina can lead to vaginal-vault prolapse, which is a fairly common condition which can be extremely debilitating. There are a number of different types of prolapse, including cystocele, where the bladder falls back onto the anterior vaginal wall. This often results in a distortion of the urethra and as a result, continence problems. Another form of pelvic organ prolapse is called a Rectocele, in which the large intestine collapses forwardly onto posterior wall of the vagina. This condition is also associated with continence problems (fecal and/or urinal).

There are a number of treatments available for pelvic organ prolapse including vaginal surgery to ‘tighten’ or narrow the vagina, so as to re-support the weakened part of the vaginal vault and re-position the prolapsing organ back away from the vagina.

More common methods of treatment include surgery to implant a mesh supporting the bladder (cystocele) or rectum (rectocele). The mesh creates a layer of fibrous or scar tissue that secures the mesh and strengthens the weakened section of the vaginal vault.

Effecting pelvic organ prolapse repairs sometimes uncovers ‘occult’ incontinence that has not been clinically apparent while in the simultaneous presence of the pelvic organ prolapse. For this reason simultaneous prolapse repairs and incontinence procedures are often completed during the same surgery.

Female incontinence can be surgically addressed in a variety of techniques. The most common method of surgical treatment of incontinence involves the use of various types of “slings” the majority of which are made from various artificial mesh-type products.

One sling technique involves implanting a sling under the middle section of the urethra (that is, above the anterior vaginal wall). The type of mesh material resembles shark skin and so ‘catches’ and is held by the tissue, preventing the material slipping from its intended position. Typically these slings are placed so as to avoid tensioning the urethra (these are collectively referred to as “tension-free slings”). If intra-abdominal pressure increases and passes down into the pelvis causing movement of the pelvic organs, the resulting movement of the bladder neck or adjacent pelvic can cause the involuntary loss of urine in women. The mid-urethral tension-free sling acts as a ‘backboard’ under the urethra and limits the extent of movement of the urethra, temporarily compressing it and preventing the loss of urine. In some instances, tension-free slings actually move through a fulcrum when intra-abdominal pressure increases are passed down into the pelvis. This “fulcrum” movement serves to ‘kink’ the urethra and prevent loss of urine.

Another type is a pubo-vaginal sling (PV sling) which is proximally placed near to the bladder neck and in this instance, using tension to “pull up” the sling behind the pubic bone and to elevate the bladder neck. Increased intra-abdominal pressure that is passed into the pelvis then clamps down on the elevated, immobilised urethra.

The use of “tightening” procedures as well as the newer mesh-based surgeries have varying levels of success in correcting vaginal prolapse. A long time is generally required for healing the repair to the prolapse. Further, if not addressed at the same time as the prolapse repair, incontinence problems may emerge or in some cases be exacerbated—for example in the case where a woman's bladder collapse is corrected, the consequent straightening of the urethra may result in a previously hidden continence problem surfacing but one in which the complex supporting musculature surrounding the urethra is now weakened. Stress Urinary Incontinence (SUI) in females manifests in one of two ways: firstly as hypermobility of the bladder, usually inferring both descent and rotation of the bladder (greater than 2 cm from its normal resting position) resulting in the above-mentioned urethral distortion, and secondly, intrinsic sphincter deficiency (or ISD), which infers the innate failure of the muscular complex that surrounds the female urethra and serves to coapt or close the urethra and prevent accidental loss of urine. Many women have a combination of these two types of ‘stress urinary incontinence’ which can result in leakage as a result of stress events such as coughing, laughing etc.

In an earlier patent application, International patent application no. PCT/AU00/0095 (the disclosure of which is incorporated herein by reference), a method and apparatus is proposed for treating urinary incontinence which includes the steps of forming a “neosphincter” from smooth muscle tissue taken from elsewhere in the patient's body, and wrapping the neosphincter around the urethra. An implantable stimulator provides an electrical signal to the neosphincter via an electrode or electrodes. The electrical signal stimulates the neosphincter to maintain tone about the urethra to reduce leaks from the bladder until the user wishes to urinate. A signal from a control device may cause the stimulator to stop providing electrical signals to the neosphincter, to allow the neosphincter to relax and enable the individual to urinate. The stimulation may activate the muscle directly, or through the excitation of nerve fibres that innervate the muscle.

### SUMMARY OF THE INVENTION

In accordance with a first aspect, the present invention provides an apparatus for treating a condition associated with vaginal prolapse, the apparatus including an implantable stimulator arranged to be implanted within a patient and to provide a signal for stimulation of tissue in the pelvic region, in order to treat the condition.

In an embodiment, the condition is associated with corrective surgery which has been carried out to treat the prolapse.

In an embodiment, the implantable stimulator may be arranged to provide a signal to contractile tissue which is positioned about the urethra in order to contract the urethra and maintain continence. The contractile tissue may be a smooth muscle sphincter, as described in the above referenced PCT application. Alternatively, the contractile tissue may be a smooth muscle sling which partially surrounds the urethra. Mesh or some other flexible, biocompatible material may also be provided to support the sling or sphincter and urethra and/or bladder. In an embodiment, the sling or sphincter may be cradled by a mesh sling. Electrodes may be provided on the mesh to stimulate surrounding tissue in the pelvic area. The contractile tissue may be implanted smooth muscle tissue or skeletal muscle tissue.

In an embodiment, the stimulator is arranged to stimulate contractile tissue to cause the tissue to contract to treat the condition. In an embodiment, the contractile tissue may be implanted and arranged to elevate and support the bladder on contraction in order to facilitate urinary continence. One or more electrodes may be positioned to enable the stimulator to stimulate the contractile tissue.

In an embodiment, where a smooth muscle sling or sphincter is stimulated to maintain coaptation of the urethra, in an embodiment a relatively inflexible support may be provided to contact the sphincter or sling and provide support therefore. When stimulation is applied to the sphincter or sling, the contractile tissue is squeezed against the support to vary the tension to the urethra.

The term “relatively inflexible” refers to a support that can move with the pelvic organs, but at a point of raised intra-abdominal pressure, is inflexible enough to provide the requisite “backboard” to enable the desired variation in tension on contraction of the contractile tissue. The support may be provided by a flexible yet inelastic mesh sling which, in an embodiment cradles the sphincter or contractile tissue. The support may mount electrodes to stimulate other tissue in the pelvic region.

In an embodiment, mesh is provided to provide further support to the bladder. In an embodiment, electrodes may be provided on the mesh to stimulate surrounding pelvic tissue. The mesh may mount one or more electrodes. In an embodiment, the mesh itself may be coated or treated in some manner so that the mesh is electrically conductive yet retains similar mechanical properties (“active mesh”), reducing the risk of fatigue points which occur when there are areas of mesh different mechanical properties immediately adjacent to each other. The contractile tissue may be implanted smooth muscle tissue or skeletal muscle tissue.

In an embodiment, the contractile tissue may be formed as a sling about a portion of the urethra to support the urethra. A portion of the sling may be mounted to other parts of the pelvic anatomy, such as the pubic bone.

The device may further include a support for the contractile tissue. In an embodiment, the support is in the form of a relatively inflexible mesh. In an embodiment, the mesh is arranged to cradle the contractile tissue. Advantageously, in operation, the contractile tissue contracts against the relatively inflexible support which provides supplementary tension to the bladder and/or urethra when there is sudden changes in intra-abdominal pressure due to a stress event (for example, a cough or sneeze) or at other times.

Australian Provisional patent application number 2005905673, the disclosure of which is incorporated herein by reference in its entirety, discloses a method and apparatus for treating fecal incontinence, which includes the step of stimulating a contractile tissue sphincter which is implanted around the colon and/or rectum in the anal region.

In an embodiment of the present invention, the implantable stimulator may be arranged to provide a signal to contractile tissue which is positioned about the colon and/or rectum in the anal region in order to maintain fecal continence. This is advantageous where, for example the prolapse condition has affected fecal continence. A mesh arrangement may also be provided to support the colon and/or rectum in the fecal area. The mesh arrangement may cradle the contractile tissue.

In an embodiment, the stimulator is arranged to stimulate an area which has been reinforced by mesh or a sling during surgery to correct the prolapse, in order to promote healing. One or more electrodes may be positioned so that the stimulator can provide signals to the area.

In an embodiment, the stimulator is arranged to stimulate the wall of the vagina or adjacent pelvic tissue outside of the vaginal vault in order to facilitate healing and tightening of the wall of the vagina following surgery for prolapse. One or more electrodes may be placed in the area in order for the stimulator to be able to provide the stimulation.

In an embodiment, the stimulator is arranged for stimulation of any one of a number of pelvic floor muscles including (but not limited to) the coccygeus muscle, pubococcygeus muscle, the iliococcygeus muscle, the sacrococcygeus muscle, the puborectalis muscle and the levator ani muscle. One or more electrodes may be placed to enable the stimulation.

In the embodiments where the stimulator is arranged to stimulate an area to promote healing, the wall of the vagina, pelvic floor muscles or other areas of the pelvic anatomy, the electrodes may be discretely placed and/or mounted by mesh which is in place to reinforce the pelvic region or incorporated as electrically conductive areas of the mesh (“active mesh”).

The implantable stimulator may be arranged to deliver signals to contractile tissue about the urethra and/or colon and/or rectum as discussed above in previous embodiments as well as one or more signals to other areas of the pelvic anatomy in order to promote healing, stimulate the wall of the vagina, stimulate one or more pelvic floor muscles or other areas of the pelvic anatomy.

In any one or more of the above embodiments a sensor or sensors may be provided for sensing a stress event, such as a laugh, cough or other stress event. In response to the stress event, the stimulation provided by the stimulator may be varied. For example, in the embodiment where implanted contractile tissue is utilised to pull up and support the bladder, following the occurrence of a stress event stimulation may be applied to cause the contractile tissue to contract and support the bladder.

The implantable stimulator may produce a plurality of separate signals or a single electrical signal of a predetermined pattern, in order to implement two or more of the above discussed embodiments. For example, a single electrical signal at a predetermined pattern may be used to stimulate a contractile sphincter about the urethra where one is implanted and also to stimulate (via other electrodes placed in different positions) other areas of the pelvic anatomy to treat the conditions associated with the prolapse.

In accordance with a second aspect, the present invention provides a device for treating a condition associated with vaginal prolapse, the device comprising contractile tissue positioned proximate to the bladder and/or urethra and arranged to be stimulated to contract to provide support to the bladder/urethra.

The contractile tissue may include one or more implantable electrodes for providing electrical stimulation.

In one embodiment the contractile tissue is smooth muscle tissue.

In one embodiment the contractile tissue is skeletal muscle tissue.

In one embodiment the contractile tissue is formed into a sling arranged to pull up the urethra.

In accordance with a third aspect, the present invention provides a controller for controlling an implantable stimulator which is arranged to stimulate tissue to treat a condition associated with vaginal prolapse, the controller including means for providing a signal to the implantable stimulator to vary the stimulation provided by the implantable stimulator.

In one embodiment, the controller may include means for varying the amplitude and/or frequency of stimulation in order to vary the effect.

In accordance with a fourth aspect, the present invention provides a system for treating a condition associated with vaginal prolapse, the system comprising an apparatus in accordance with the first aspect of the present invention, and a device in accordance with the second aspect of the invention.

In accordance with a fifth aspect, the present invention provides a system for treating a condition associated with vaginal prolapse, comprising an apparatus in accordance with the first aspect of the invention and a controller in accordance with the third aspect of the invention.

In an embodiment, the system also comprises a device in accordance with the second aspect of the invention.

In an embodiment, the system also includes a programmer unit which may be used to programme parameters of the implantable stimulator. For example, the programmer unit may be used to programme the signal characteristics of the stimulator signal(s). The programmer unit may be used by a medical operative, for example a physician, in order to programme parameters of the implantable stimulator. In an embodiment, the stimulator includes a communication arrangement for communicating with the programmer unit. The communication arrangement may be arranged to communicate to the programmer unit telemetry information about the operation of the stimulator, which the physician can utilise when programming the stimulator.

In accordance with a sixth aspect, the present invention provides a programmer unit for programming parameters of a stimulator unit in accordance with the first aspect of the invention.

In accordance with a seventh aspect, the present invention provides a system for treating a condition associated with vaginal prolapse, the system including an apparatus in accordance with a first aspect of the invention and a programmer unit in accordance with the sixth aspect.

In accordance with a eighth aspect, the present invention provides an apparatus for support of an implanted contractile tissue arrangement which can be stimulated electrically to maintain urinary incontinence by affecting the urethra of a patient and/or bladder of a patient, the apparatus comprising a support which is arranged in use to bear against the contractile tissue.

In an embodiment, the support is flexible yet inelastic when stretched, so that when there is a stress event, the pelvic anatomy can still move, yet still enable the contractile tissue when stimulated to bear against the support and vary tension on the urethra and/or bladder of the patient.

In an embodiment the support is a mesh support.

In an embodiment the support is in the form of a sling.

In an embodiment, the support is arranged to cradle the contractile tissue.

In an embodiment, the contractile tissue is in the form of a sling which surrounds a portion of the urethra or bladder.

In an embodiment, the contractile tissue is in the form of a sphincter.

In an embodiment, the contractile tissue is smooth muscle tissue.

In an embodiment, the contractile tissue is skeletal muscle.

In an embodiment the support mounts electrodes for stimulating tissue in the pelvic region.

In accordance with the ninth aspect, the present invention provides a system for maintaining urinary continence which comprises an apparatus in accordance with the sixth aspect of the invention and a contractile tissue arrangement which is stimulated electrically to maintain urinary continence by affecting the urethra of a patient and/or the bladder of a patient.

In accordance with an tenth aspect, the present invention provides an apparatus for supporting an implanted contractile tissue arrangement which is stimulated electrically to maintain fecal incontinence by affecting the colon of a patient, the apparatus comprising a support which is arranged in use to bear against the contractile tissue.

In an embodiment, this support is relatively inflexible so that when the contractile tissue bears against it tension on the colon is varied.

In an embodiment, the support is a mesh support.

In an embodiment, the support is a sling which is arranged to cradle the contractile tissue.

In an embodiment, the support and its electrodes arranged to stimulate tissue in the pelvic region.

In an embodiment, the contractile tissue is a sphincter.

In an embodiment, the contractile tissue is smooth muscle tissue.

In an embodiment, the contractile tissue is skeletal muscle.

In accordance with a eleventh aspect, the present invention provides a system for maintaining fecal continence, comprising an apparatus in accordance with the eighth aspect, and a contractile tissue arrangement arranged to be implanted about the colon and/or rectum and to be stimulated to maintain fecal continence by affecting the colon and/or rectum.

In accordance with twelfth aspect, the present invention provides a method of treating a condition associated with vaginal prolapse, the method comprising the steps of providing electrical stimulation to the pelvic anatomy to treat conditions associated with vaginal prolapse.

In an embodiment, the condition is associated with corrective surgery which is being carried out to treat the prolapse.

In an embodiment, the step of providing electrical stimulation comprises the step of providing a signal to contractile tissue which is conditioned about the urethra in order to contract the urethra and maintain continence. The contractile tissue may be a smooth muscle sphincter as described in the above referenced PCT Application (PCT/AU00/00095). Alternatively, the contractile tissue may be a smooth muscle sling which partially surrounds the urethra. Mesh may also be provided to support the sling and/or sphincter and urethra and/or bladder. In an embodiment the step of providing electrical stimulation comprises providing stimulation to surrounding tissue in the pelvic area via electrodes mounted in or on the mesh. The contractile tissue may be implanted smooth muscle tissue or skeletal muscle tissue. In an embodiment, the mesh may incorporate areas that are electrically conductive areas to stimulate the contractile tissue and/or the surrounding pelvic anatomy.

In an embodiment, the step of applying stimulation includes the step of applying stimulation to stimulate contractile tissue sling which is arranged to elevate and support the bladder and/or urethra.

In an embodiment, mesh is provided to further support the bladder. The step of providing electrical stimulation in an embodiment may comprise providing stimulation to surrounding pelvic tissue via electrodes mounted by the mesh.

In an embodiment, the step of applying electrical stimulation comprises the step of stimulating a contractile tissue sphincter which is implanted around the colon and/or rectum in the anal region to facilitate fecal continence. A mesh arrangement may be provided to support the colon and/or rectum in the fecal area. In an embodiment, the step of electrical stimulation comprises the step of stimulating surrounding pelvic tissue via electrodes mounted by the mesh.

In an embodiment, the step of applying electrical stimulation includes the step of applying electrical stimulation to an area which has been treated by surgery to implant a support for supporting organs to correct the prolapse, in order to promote healing. The support may be a mesh or a sling for supporting the bladder or uterus or the colon or the rectum, or any combination of these.

In an embodiment, the step of applying stimulation includes applying stimulation to the wall of the vagina in order to facilitate tightening of the vaginal wall following surgery for the correction of prolapse.

The method may include the further step of detecting an increase in abdominal pressure, such as may be brought about by a “event” such as a laugh or a cough, and applying stimulation to the contractile tissue in response to the increased pressure.

In an embodiment, the method may also include the step of implanting the contractile tissue sling and/or sphincter.

In an embodiment, the step of stimulation may include stimulation of muscles lining the length of the urethra.

In an embodiment, the step of stimulation may include stimulating the various muscles of the pelvic floor. Stimulation may be applied to the coccygeus muscle, pubococcygeus muscle, the iliococcygeus muscle, the sacrococcygeus muscle, the puborectalis muscle or the levator ani muscle (or any combination of the above named muscles/muscle groups within the pelvic floor).

In accordance with a thirteenth aspect, the present invention provides a method of treating a condition associated with vaginal prolapse, comprising the step of implanting into a patient a stimulator device arranged to provide stimulation signals to treat the condition.

In an embodiment, the method also includes the step of implanting contractile tissue sphincter about the urethra. The sphincter being arranged to be stimulated to maintain coaptation of the urethra.

In an embodiment, the method also includes the step of implanting the contractile tissue sphincter about the colon and/or rectum in the anal region. The contractile tissue sphincter being arranged to be stimulated in order to facilitate fecal continence.

In an embodiment, the method also includes the step of implanting contractile tissue, the contractile tissue being implanted in position such that when it is stimulated it will elevate and support the base of the bladder and/or the urethra.

In an embodiment, the method also includes the step of implanting an electrode or electrodes in or on the contractile tissue so that the stimulator device may provide stimulation to the contractile tissue.

In an embodiment, the method also includes the step of implanting mesh. The embodiments which include the steps of implanting contractile tissue, the method may also include the step of implanting a support supporting the contractile tissue. The support may be in the form of a mesh. The support may mount electrodes for stimulating tissue in the pelvic region.

In an embodiment, the method includes the step of implanting electrodes in the pelvic anatomy so that stimulation signals can be provided via the electrodes.

In an embodiment, the method also includes the step of implanting one or more sensors for sensing a stress event, such as a laugh, cough or other stress event.

In accordance with a fourteenth aspect, the present invention provides a method of treating a condition associated with vaginal prolapse, comprising the step of implanting contractile tissue in the pelvic anatomy, the contractile tissue being arranged to be stimulated to treat the condition.

In an embodiment, the contractile tissue is in the form of a sling partially surrounding the urethra and/or bladder.

In an embodiment, the contractile tissue is in the form of a contractile tissue sphincter surrounding the urethra.

In an embodiment, the contractile tissue is in the form of a contractile tissue sphincter to be implanted about the colon and/or rectum in the anal region in order to facilitate fecal continence.

### BRIEF DESCRIPTION OF THE DRAWINGS

Features and advantages of the present invention will become apparent from the following description of embodiments thereof, by way of example only, with reference to the accompanying drawings, in which:

FIG. 1 is a diagram showing the female anatomy and including an apparatus in accordance with an embodiment of the present invention implanted within the female anatomy;

FIG. 2 is a diagram showing the female anatomy and an implanted apparatus in accordance with a further embodiment of the present invention;

FIG. 3 is a diagram showing the female anatomy and an implanted apparatus in accordance with yet a further embodiment of the present invention;

FIG. 4 is a diagram showing the female anatomy and an implanted apparatus in accordance with a further embodiment of the present invention;

FIG. 5 is a diagram showing the female anatomy and an implanted apparatus in accordance with a further embodiment of the present invention;

FIG. 6 is a diagram showing the female anatomy and an implanted apparatus in accordance with a further embodiment of the present invention;

FIG. 7 is a block diagram of a stimulator apparatus in accordance with an embodiment of the present invention;

FIG. 8 is a block diagram of an apparatus in accordance with an embodiment of the present invention;

FIG. 9 is a block diagram of an apparatus in accordance with an embodiment of the present invention;

FIG. 10 is a block diagram of a stimulator in accordance with an embodiment of the present invention;

FIG. 11 is a block diagram of an apparatus in accordance with an embodiment of the present invention;

FIG. 12 is a block diagram of an apparatus in accordance with an embodiment of the present invention;

FIG. 13 is a diagram showing the female anatomy and an implanted apparatus in accordance with an embodiment of the present invention.

FIG. 14 shows an apparatus in accordance with an embodiment of the present invention.

FIG. 15 shows a diagram of the female anatomy and an apparatus in accordance with yet a further embodiment of the present invention.

### DETAILED DESCRIPTION OF EMBODIMENTS

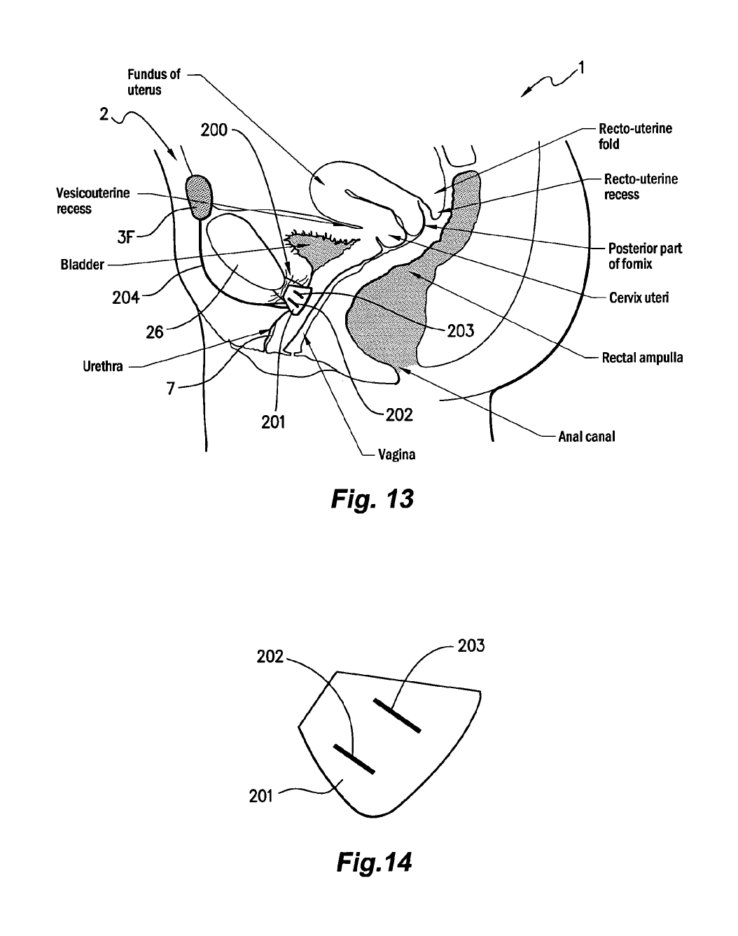

Referring to FIG. 13, a schematic diagram showing the female pelvic anatomy is shown, generally designated by reference numeral 1. An apparatus in accordance with an embodiment of the present invention, generally designated by reference numeral 2 is implanted within the female pelvic anatomy. The apparatus 2 includes an implantable stimulator 3F, which in this example includes a signal generator arranged to provide a signal to stimulate contractile tissue 200 which is positioned about the urethra 7.

In this embodiment, the contractile tissue 200 is in the form of a sling which loops around the back of the urethra 7 and has ends attached to the pubic bone 26.

In alternative embodiment, the contractile tissue 200 may be in the form of a sphincter completely surrounding the urethra 7, similar to the sphincter disclosed in the above-referenced PCT Application.

In this example, the contractile tissue sling 200 is of smooth muscle tissue. It may be of other contractile tissue type, however, such as skeletal muscle.

In this embodiment, under stimulation the smooth muscle sling 200 operates to provide tension in the direction of the pubic bone and close the urethra, to maintain continence. When the patient wishes to urinate, a provided controller (see later) is actuated to enable the signal to be changed or switched off entirely, in order to enable the smooth muscle sling 200 to relax, allowing the urethra 7 to open and the patient to urinate.

In an alternative embodiment utilising a sphincter instead of a sling, as the contractile tissue, a similar process may be applied for continence and urination.

It is believed that, particularly in the female patient, there may be some advantages of utilising a contractile muscle sling, as it may be easier to implant surgically.

In this embodiment, in addition to the contractile tissue 200, a support 201 is provided for supporting the contractile tissue 200. In this embodiment, the support 201, is in the form of a mesh sling which cradles the contractile tissue 200. The mesh sling 201 provides a relatively rigid “backboard” against which the contractile tissue sling (or sphincter in the alternative embodiment) may contract. In addition, the support may assist the surgeon in placing the contractile tissue, backed by the support, in the vicinity of the urethra. Stimulation can vary the tension of the urethra. The mesh acts as a backboard against which the contractile tissue 200 squeezes as it is stimulated, increasing the effect on the closing of the urethra.

The sling 201 may therefore be readily adjusted by varying the level of stimulation from the stimulator 3F. As there are variations over time neither urethral anatomy (for example, fibrosis or some other changing physical form) bladder function, pelvic floor strength (or variations thereof), stimulation may be adjusted to provide superior function.

In this embodiment, the mesh sling 201 also mounts electrodes 202, 203. These electrodes may be used to stimulate the surrounding pelvic area. For example, for training and building strength of the pelvic tissue to facilitate support of the urethra and bladder. The electrodes may also be used to respond to stress events, such as a laugh or a cough of the patient. In response to such a stress event, the electrodes may stimulate skeletal muscle in the area in order to contract and provide additional support to the bladder and/or urethra. A sensor arrangement may be incorporated in the stimulator 3F to detect increase in pressure in the abdominal area such as may occur because of a stress event. Sensors may be incorporated elsewhere in the abdominal area if required.

Conductive insulated leads 204 transmit signals from the stimulator 3F to the contractile tissue 200 and mesh sling mounted electrodes 202, 203.

Other electrodes may be placed in other positions within the pelvic area to stimulate other tissue, in accordance with the embodiments that are discussed in the following, which may be used in combination with the embodiment of FIG. 13.

The embodiment of FIG. 13 may be used to treat a condition associated with vaginal prolapse, in order to support the bladder and urethra and maintain continence. The embodiment is not limited to the treatment of the condition associated with vagina prolapse, however, and may be used treat any incontinence problem, even those which are not associated with vaginal prolapse.

Note that after initial surgical implantation of the sling 200 (or sphincter in the alternative embodiment), the stimulator 3F may be arranged to provide low level electrical stimulation to the neo-sphincter either via implanted electrodes or electrodes on mesh sling 201, in order to facilitate renovation of the contractile tissue following the surgery.

FIG. 14 shows the mesh sling 202 in more detail.

Inelastic “back board” mesh sling that cradles smooth muscle graft
natural smooth muscle in contact with urethra
sling configuration requires less invasive surgery than circumferential wrap
additional stimulation intensity can increase activation of smooth muscle, increasing tension on urethra.
stimulation off during voiding reduces obstructive effect of sling.

Conductive electrode areas on back of mesh
allows optional stimulation of surrounding pelvic floor for training and building strength;
may allow dynamic, active adjustment of pelvic floor musculature e.g. rapid response of skeletal muscle to cough or laugh (e.g. less than 1 second), compared with slower response of smooth muscle sphincter (several seconds);
similar “electrically active” elements could be incorporate in mesh that is used to repair cystocele or rectocele to provide diffuse pelvic floor stimulation.

The stimulation to the contractile sling and/or sphincter, and/or electrodes mounted in the mesh support and/or other electrodes mounted elsewhere in the pelvic area may be provided in a number of ways to provide a number of functions, for example:
a) To provide an initial stimulus regime that may assist wound repair following surgery. For example, if there has been surgery to repair the vagina, electrical stimulation may be provided to facilitate wound repair, for example via electrodes mounted to a mesh sling such as mesh sling 201 or other electrodes.
b) Stimulation may be varied to a smooth muscle sphincter or smooth muscle sling (such as in the embodiment of FIG. 13 (to provide adjustment in response to abdominal pressure changes, such as a sudden increase because of a stress event such as a cough or laugh (as discussed both) and/or a progressive increase in stimulation due in the straining.

Referring to FIG. 1, in this embodiment, conductive leads (which are insulated) conduct signals to various implanted electrodes, implanted in the pelvic anatomy, in order to stimulate tissue within the pelvic anatomy to treat a condition associated with vaginal prolapse.

In more detail, the pelvic anatomy illustrated in the FIG. 1 diagram includes the uterus 4, the bladder 5, the vagina 6, the urethra 7, and the rectum 8.

This diagram illustrates treatment which has been applied to treat a cystocele vaginal prolapse. Mesh 9 has been surgically implanted to support the bladder 5 which had collapsed back on to the vagina 6. The mesh 9 raises the bladder 5 to ease pressure on the vagina 6. This is a known treatment.

In accordance with this embodiment, stimulation is provided by the stimulator 9 to stimulate tissue in order to facilitate healing following implantation of the mesh 9. Electrodes 10 and 11 positioned close to or at the mesh are stimulated by an appropriate signal which will promote healing of the tissue around the mesh. Stimulation may be to sensory or motor nerves within the tissues or to the tissues themselves in order to promote the healing.

Electrode 12 and also electrode 11 are arranged to provide signals to stimulate the wall of the vagina in order to facilitate healing and tightening of the wall of the vagina. Electrodes 13 and 14 are arranged to stimulate other areas of the pelvic anatomy, such as the pubococcygeus muscle, the levator ani muscle or muscles found within the pelvic floor muscular complex.

Note that electrodes may be placed in other parts of the patient's anatomy to treat and promote healing as appropriate.

The stimulator may produce a single signal or a number of different signals going to different electrodes. Appropriate signal patterns may be implemented in order to provide the appropriate stimulation. In this embodiment, parameters of the stimulating signal produced by the stimulator 3 are variable, to enable adjustment to the stimulus, as will be discussed in more detail later.

The stimulator 3 is also arranged to enable the stimulation to be turned off, to one or more or all electrodes, at intervals or under external control, e.g. by a medical operative or a user of the apparatus.

In a further embodiment, the mesh 9 may mount electrodes (eg. Electrodes 10 and 11 may be mounted by the mesh) to provide the stimulation. Preparing mesh that incorporates its own electrodes (“active mesh”) may have advantages in reducing the amount of surgery to implant electrodes into the pelvic region and provide a superior mesh that has consistent mechanical properties, reducing the risk of fatigue of the mesh in the area adjacent to the stimulation electrodes. As a further embodiment that is simpler to implant surgically, mesh which incorporates an array of electrodes allows selection of the site(s) of electrical stimulation at different areas of the mesh after implant to provide the intended appropriate effect of that stimulation.

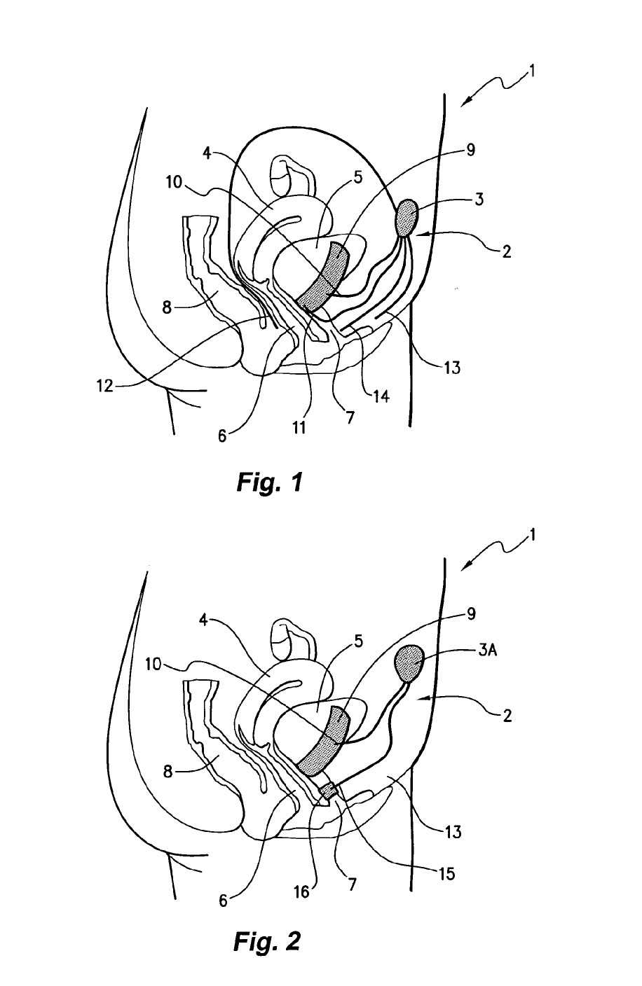

A further embodiment of apparatus in accordance with the present invention is illustrated in FIG. 2. The same reference numerals have been used in FIG. 2 to identify elements the same as those in FIG. 1, and no further description will be given here.

Reference numeral 3A illustrates an implanted stimulator in accordance with this embodiment. The stimulator is arranged to provide a signal to an electrode 10, which signal is arranged to promote healing of tissue associated with a mesh implant 9 (to correct a cystocele vaginal prolapse). This is similar to electrode 10 discussed in relation to FIG. 1.

In FIG. 2, a contractile tissue neosphincter is shown surgically implanted around the urethra 7. The neosphincter has been implanted in accordance with the disclosure of the above-referenced PCT application (PCT/AU00/00925). A stimulator 3A provides a signal via electrodes 15 to cause the contractile tissue neosphincter 16 to contract and constrict the urethra 7, maintaining urine in the bladder 5. In accordance with the disclosure of the above-numbered PCT application, a further signal (or absence of signal) may be generated by the stimulator 3A in order to enable the sphincter 16 to relax and allow the patient to urinate.

In this embodiment, the neosphincter is a smooth muscle neosphincter. The smooth muscle sphincter will usually have been taken from elsewhere in the body and transplanted, as taught in the referenced PCT application. The smooth muscle may alternatively be grown tissue, or smooth muscle taken from the body reinforced with grown tissue.

In the embodiment of FIG. 2, therefore, electrical signals are provided to promote healing and strengthen the area around the mesh implant, as well as at the same time controlling continence by stimulation of the neosphincter 16. Note that in this embodiment, electrodes could be placed elsewhere in the pelvic anatomy, as disclosed in relation to FIG. 1, and stimulated to promote healing and strengthen other areas of the pelvic anatomy, in order to treat the condition.

Note that the smooth muscle sphincter may be replaced by a smooth muscle sling, as in accordance with the embodiment of FIG. 13, and the mesh may be “active” mesh in corporating electrodes used for stimulation.

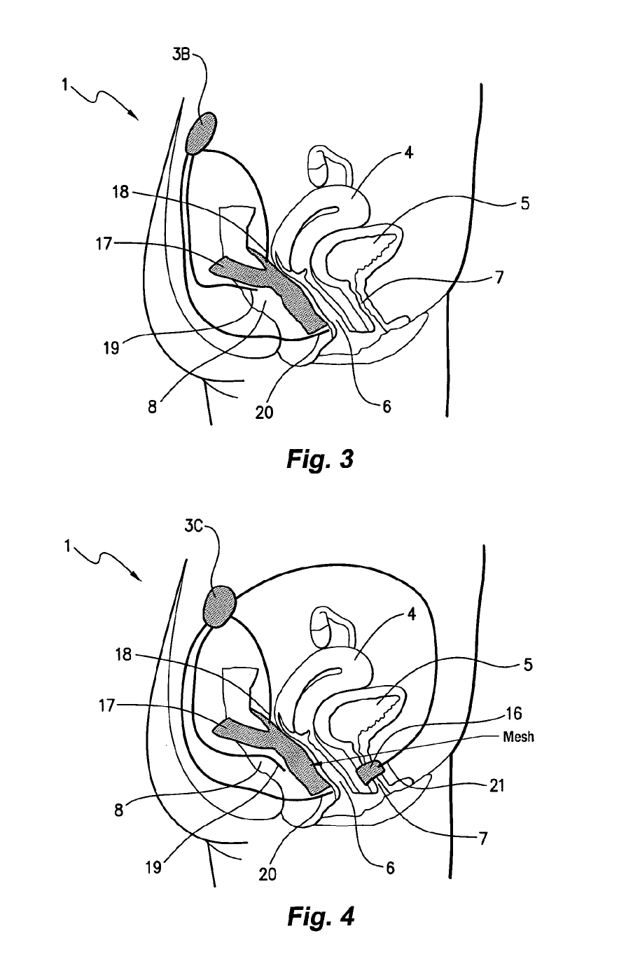

A further embodiment of the present invention is illustrated in FIG. 3, implanted in the female anatomy. Again, the same reference numerals have been used for the same elements and no further description will be given.

FIG. 3 shows a representation of corrective surgery to treat a rectocele vaginal prolapse. The rectum 8 is supported by a mesh support 17, implanted to support the collapsed rectum 8 away from the vagina 6.

Stimulator 3B is arranged to provide signals to electrodes 18 and 19 in order to promote healing of the tissue about the mesh 17 and facilitate strengthening of the tissue in that area. In addition, a further electrode 20 is arranged for stimulation of the rear of the vaginal wall (the sacrococcygeus muscle) to promote healing and tightening of the vaginal wall.

Note that electrodes may be placed within the other areas of the pelvic region as appropriate. Electrodes may be incorporated in “active mesh”.

Stimulation in this embodiment will be as appropriate to promote the tissue healing and strength.

FIG. 4 shows a variation of the embodiment of FIG. 3. Again, the same reference numerals have been used for similar elements. In this embodiment, stimulator 3C is arranged to provide stimulation signals to electrodes 18, 19 and 20, for a similar function to the FIG. 3 embodiment, and in addition to a further electrode 21 for stimulation of a smooth muscle neosphincter 16 wrapped about the urethra 7. The smooth muscle sphincter has a similar function to the smooth muscle sphincter 16 referred to in FIG. 2 above. Stimulator 3C is arranged to provide a stimulation signal to cause the smooth muscle sphincter 16 to contract, constricting the urethra 7 retaining urine in the bladder 5. A further signal (or absence of signal) may be transmitted to cause the smooth muscle sphincter 16 to relax and allow urine to flow.

This embodiment therefore treats a combination of rectocele vaginal prolapse and incontinence, which may have been brought on by the vaginal prolapse or the treatment for the vaginal prolapse.

A contractile tissue sling may be used in place of the neo sphincter.

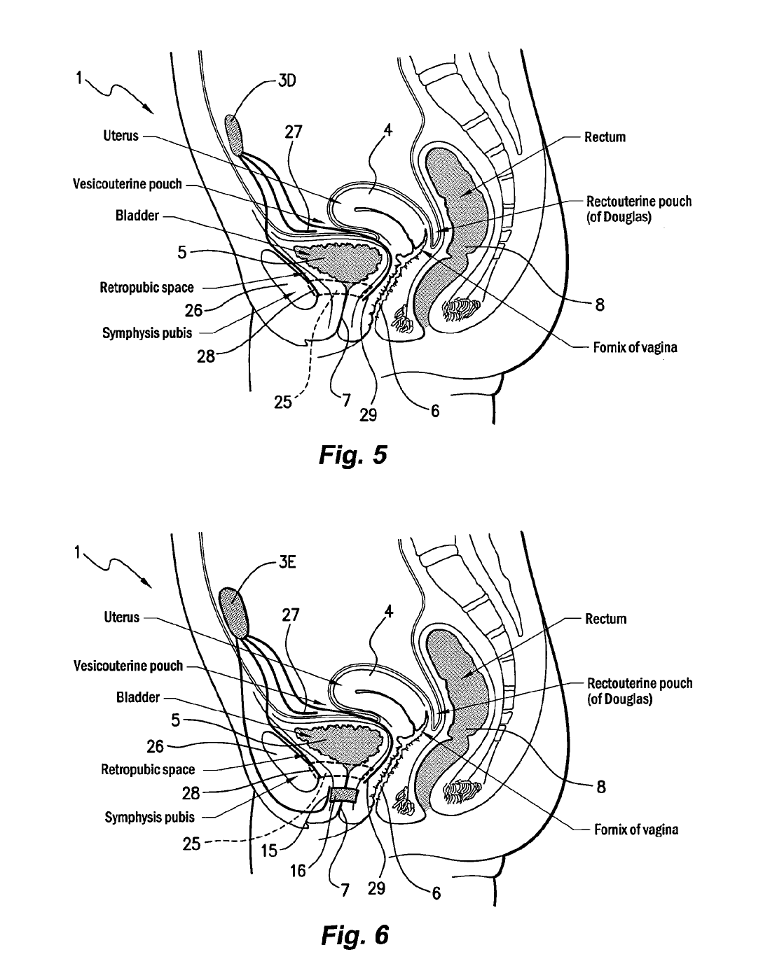

FIG. 5 illustrates yet a further embodiment of the present invention. The same elements have been given the same reference numerals as in previous embodiments and no further description will be given here. In this embodiment, a contractile tissue sling has been implanted to support the bladder. The sling is designated by reference numeral 25 and shown in ghost outline. The sling attaches to the pubic bone 26, wraps around the back of the bladder 5 and back to the pubic bone 26, so as to form a support for the bladder 5 secured by the pubic bone. Note that, although not shown here, mesh may also be included to support the bladder away from the vagina. The mesh may not be essential however.

This arrangement may be used for treatment of a vaginal prolapse (cystocele) and/or treatment of incontinence.

In this embodiment, a receptor 27 is mounted to the bladder. The receptor 27 is a pressure sensor which is arranged to monitor for changes in pressure within the abdomen. Note that although it is shown mounted to the bladder 5 in this embodiment, it may be mounted elsewhere in the abdomen if other positions will provide a better reading of variations in pressure. Signals from the sensor 27 are fed back to the stimulator 3D and utilised by a processor mounted in the stimulator 3D to control the stimulation applied to the sling 25 via further electrodes 28 and 29.

In this embodiment, on an increase in pressure within the abdomen, such as may be caused by a person laughing or coughing, this is detected by the sensor 27, and utilised by the stimulator 3D to provide or vary signals to the sling 25 in order to cause the sling to contract. The contracting causes the bladder 5 to be “pulled up” and facilitate maintenance of continence in response to these “stress” events (coughing, laughing etc).

In addition or alternatively to varying stimulation when a stress event occurs, stimulation may be varied to keep the bladder pulled up and to keep up and to keep the bladder pulled up during straining, etc.

The contractile tissue sling 25 may be of any type of contractile tissue, and may be skeletal muscle and/or smooth muscle.

The sling and stimulator arrangement therefore together form a “dynamic sling” which reinforces continence during stress events or generally.

A further embodiment is shown in FIG. 6. This embodiment includes the same features of a “dynamic sling” 25 of the embodiment of FIG. 5. The same reference numerals have been used for the same elements and no further description will be given.

In this embodiment, a smooth muscle neosphincter has been implanted about the urethra 7 and is stimulated by electrode 15 to further facilitate continence. The smooth muscle neosphincter 16 is implanted in accordance with the disclosure given in the above referenced PCT application.

In this embodiment, the patient is provided with the benefits of both the “dynamic sling” responsive to stress events, and the neosphincter 16 to control opening and closing of the urethra 7. Note that stimulation to the neosphincter 16 may be further controlled in response to a stress event detected by sensor 27. Note that a contractile tissue sling(as in the embodiment of FIG. 13) may be used in place of the neosphincter.

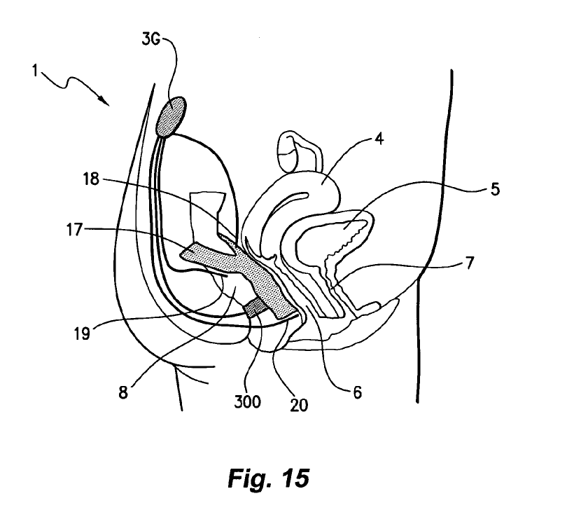

Yet a further embodiment of the present invention is illustrated in FIG. 15. This embodiment is a modification of the embodiment of FIG. 3, and the same reference numerals have been used for the same elements and no further description would be given.

As discussed in the pre-amble of this document, the applicants have lodged an earlier provisional Patent Application, Australian Patent Application No. 2005905673, which discloses implant of a contractile tissues sphincter about the colon and/or rectum in the anal region, in order to facilitate fecal continence.

In the embodiment of FIG. 15, in order to deal with problems with fecal continence, a contractile tissue sphincter 300 has been implanted about the rectum. The contractile tissue sphincter may be smooth muscle tissue, as in the above numbered provisional Patent Application.

Stimulation is provided by stimulator 3G to electrodes implanted in the smooth muscle sphincter 300 in order to maintain fecal continence. As with the embodiment of FIG. 3, electrodes that are placed elsewhere in the anatomy to provide various functions for electrodes. Electrodes 18 and 19 are provided in order to promote healing of the tissue about the mesh 17 and facilitate strengthening of the tissue in that area. In addition, further electrode 20 is arranged for stimulation of the rear of the vaginal wall to promote healing and tightening of the vaginal wall.

Electrodes may be placed within other areas of the pelvic region and/or incorporated in active mesh.

Features of any of the above embodiments may be incorporated in any of the other embodiments. For example, where mesh is involved in treatment of vaginal prolapse, stimulation of tissue about the mesh in order to promote healing may be combined with stimulation of a dynamic sling and a smooth muscle sphincter. Stimulation may be provided to any part of the anatomy or sling in any embodiment, in order to promote strength and tissue healing.

In each of the above embodiments the stimulation signal and/or pattern for stimulation signals may be varied by varying the frequency signal and/or the amplitude of the signal.

Different stimulus parameters may be required for skeletal muscle eg. The pelvic floor (eg 10-30 Hz and low amplitude e.g 0.5-5 mA) as otherwise it could be perceived as painful. It can be considered that embodiments which implement stimulation of the pelvic area and the pelvic floor are in effect providing the benefits of physiotherapy (or surface functional electrical stimulation) in a controlled manner by allowing the clinician to program a sequence of strengthening activities that can be automatically initiated, or initiated by the patient without connecting intervaginal stimulators, surface electrodes to the perineum or other locations on the body's surface etc. In addition compliance to a therapeutic regime (eg. stimulus parameters, repeats of muscle activation, duration and frequency of training) can be monitored so that a supervisory clinician would alter'the stimulus regime depending on progress and a response, and optionally logged by the implanted arrangement to confirm operation of the system.

As discussed above, broad, low level stimulation or (physiotherapy type stimulation) could be delivered in a variety of ways:
1) By dedicated electrodes.
2) Mesh incorporating electrically conductive elements placed contact the surrounding tissue.
3) Insulated mesh which incorporates electrode contacts at or near its points of anchoring.

By incorporating the latter two approaches, the extensive surgery required may be considerably reduced. Because the stimulation is delivered by a pathway within the body, lower stimulus currents may be required compared with other approaches that use surface electrodes and in addition, are for more convenient and reproducible, as the patient does not have to reapply electrodes for each session. This may increase compliance in the effectiveness of the therapy.

Because there is stimulation to be delivered in a relatively diffuse manner, and distinct from that delivered to the near sphincter or sling, a separate dedicated electrical stimulation channel may be required. Alternatively, it may be possible to enter leave stimulation such that the higher frequency stimulation required during muscle training to strengthen the pelvic floor is “overlaid” on the lower frequency stimulation used to keep the smooth muscle near sphincter innovated.

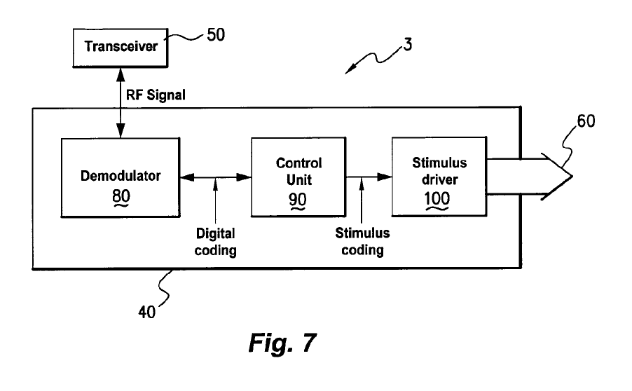

The stimulator 3 (3A, 3B, 3C, 3D, 3E) is shown in more detail in FIG. 7. In this embodiment, a signal generator means arranged to provide the electrical signal for stimulation of the sphincter or sling and of other areas of the pelvic region is in the form of a control unit 90 and stimulus driver 100. The control unit 90 encodes the stimulus and provides a signal to the stimulus driver 100 which provides the stimulation signal at output 60. The output 60 output signals to one or more conductors leading to one or more electrodes, as required for the prolapse related condition. The control unit 90 is arranged to control the stimulus driver 100 to provide one or a plurality of stimulation signals to the one or more electrodes. The control unit 90 may be differently programmed for providing the appropriate signals and/or patterns of signals to the one or more electrodes depending upon the embodiments implemented (e.g. stimulator 3, 3A, 3B, 3C, 3D, 3E, 3F, 3G or any other embodiment).

In this embodiment, the control unit 90 and stimulus driver 100 form, together with a demodulator 80, a processing unit for generating the stimulation signal(s) at output 60.

The modulator 80 is arranged to demodulate a signal received by transceiver 50. An external control unit and external programmer unit (both to be described later) are able to communicate via the transceiver with the processing unit 40 in order to control application of stimuli and/or vary the stimuli. In addition, as described in more detail later, the processing unit 40 may transmit, via control unit 90, demodulator 8 and transceiver 50, signals to the control unit or programmer unit. The transmitted signals may deliver telemetry information indicative of parameters of the stimulator, for the purposes of calibration and control.

The entire stimulator 3 (including components 40 and 50), is enclosed in a housing which includes a casing made from a bio-compatible material, such as titanium, silicone rubber or other known inert materials. The frequency of the RF signal for transmission and reception by the transceiver 50 may depend on the material of the casing of the stimulator.

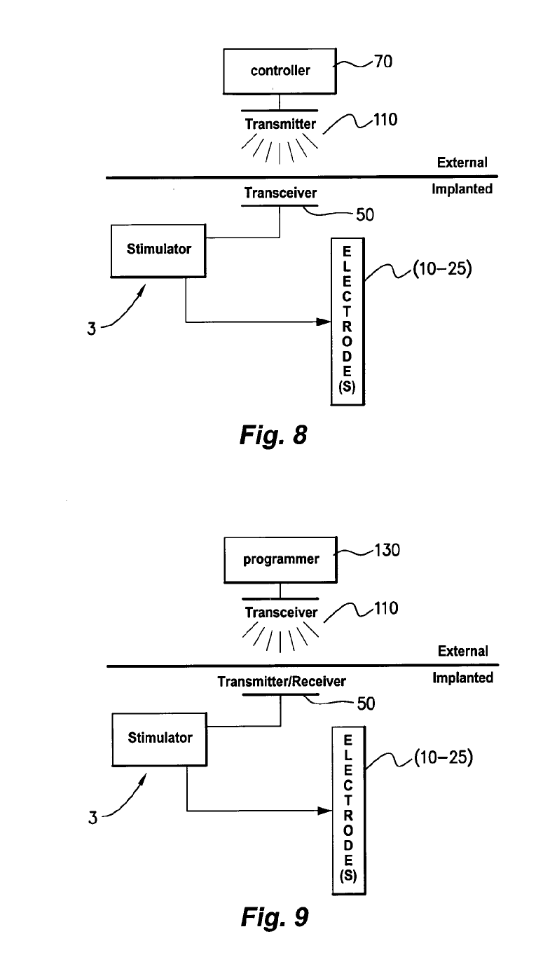

FIG. 8 shows an apparatus in accordance with an embodiment of the present invention. The apparatus incorporates the implanted stimulator 3, with transceiver 50. Electrode(s) (10-25) are shown schematically.

The apparatus also comprises an external controller 70 which includes a transmitter 110. The controller 70 is intended for operation by a patient with the stimulator implanted, for control of the stimulator 3.

The controller 70 includes means (such as a button, not shown) operable by the patient to selectively send signals to the implanted stimulator 3, for control of the stimulation signals being sent to the electrodes (10-25).

In the embodiments which include a contractile tissue sphincter or sling about the urethra, when the patient wishes to urinate, they actuate the controller 70 to send, via the transmitter 110, a signal to the stimulator. In response to receiving the signal, the control unit 90 operates to turn the stimulating signal off causing the sphincter 16 or sling to relax and allow the patient to urinate.

The controller 7 may also be arranged to provide a further signal under patient control, once the patient has finished urinating, the further signal causing stimulator 1 to resume providing the stimulation signals to the electrode(s) 15 associated with control of the neosphincter or sling 16.

The stimulation signal 6 provided to contract the sphincter 16 is selected so as to provide a substantially continuous tone in the sphincter or sling. A generally rectangular and symmetrically biphasic pulse may be suitable for this. The signal has a substantially constant current less than or equal to 50 mA, 15 mA, 10 mA, or 5 mA, and in some preferred embodiments may be in the order of 4 mA, 8 mA, 12 mA, or 15 mA. Stimulation pulse frequency provided to sphincter 1 is in the range of 0.1 Hz to 5 Hz, 0.2 Hz to 4 Hz, 0.25 Hz to 3 Hz, 1 Hz to 4 Hz, 1.5 Hz to 3 Hz, 1.75 Hz to 2.50 Hz, or 0.25 Hz to 2.5 Hz and is preferably 1 Hz, 2 Hz, 2.5 Hz, or 3 Hz. Stimulation phase width of each phase is in the range of 0.05 ms to 2 ms, 0.1 ms to 1 ms, 0.2 ms to 0.8 ms, 0.25 ms to 0.75 ms, 0.2 ms to 1.5 ms and is and is preferably 0.2 ms, 0.4 ms, 0.5 ms and 1 ms. The stimulator is current regulated, and accordingly the stimulation voltage will vary with the resistance of the muscle tissue between the electrodes. Typical values for the voltage are between 0.2 and 15 Volts, 0.2 to 10V, 0.5 to 12V, 0.5V to 10V or 0.5V to 7.5V and is preferably 2.5V, 5V, 7.5V or 10V. Either a current source (voltage limited) or a voltage source (current limited) stimulator may be used.

Signals for stimulation of tissue within the pelvic region, associated with slings or meshes or elsewhere in the pelvic region, are also provided at output 60 and sent to the appropriate electrodes (10-25). Stimulation signals may vary in frequency, pattern and amplitude, in order to provide the appropriate stimulation in each case.

The electrode structure may be any appropriate electrode structure. In one embodiment, the electrode for stimulating the neosphincter may be of the structure of the electrode which is disclosed in Applicant's co-pending PCT application number PCT/AU2005/001698, which discloses an electrode having two extending, opposing arms mounting conductive elements for stimulation of tissue, such as a neosphincter, which sits between them. The disclosure of this earlier PCT application is incorporated herein by reference.

As well as switching the stimulator 3 on and off, the controller 70 may have further patient control functions. It may, in one embodiment, include “fine” control to vary the parameters of the stimulation being applied by the stimulator 3. For example, before the patient goes to sleep, they may wish to provide a different pattern of stimulation. The controller 70 may therefore include a patient manipulator or control to enable the patient themselves to adjust the stimulation.

FIG. 9 shows an apparatus in accordance with an embodiment of the present invention, including a programmer unit 130 which may be utilised by a physician to set and adjust parameters of the implanted stimulator 3. The programmer 130 may be an appropriate means for communicating with the stimulator via transceiver 110, and may include a computing device. The control unit 90 is also arranged to transmit stimulator telemetry information indicative of one or more of the parameters of the stimulator 3, for detection by the programmer 130 via transceiver 110. The programmer unit 130 can therefore determine parameters of the stimulator from telemetry information and can adjust the parameters by transmitting control signals to the stimulator 3. The signal from the programmer may be able to selectively vary the output current, shape, frequency and/or pulse width of the stimulation signal(s).

In operation, a physician adjusts parameters of the stimulation signal(s). The physician will note feedback from the patient as to the effect of the stimulus on bladder control, and may subsequently re-adjust the parameters until the stimulation is optimum. For example, patient perceived feedback may be used to set the maximum stimulation threshold of the smooth muscle sphincter (for example, any overflow stimulation to the neosphincter may elicit and/or be perceived as an urgency event by the patient).

In the above-described embodiments, signals between the controller or programmer and the stimulator are RF signals. Other types of transmission media other than RF may be used. For example, microwave signals may be used for transmission, optical signals may be used, and in another embodiment magnetic transmission may be used.

Magnetic transmission may be used for the controller unit 70 to cause the stimulator to stop producing stimulation signals and therefore allow the patient to urinate. In this embodiment, the control unit 70 may be a simple magnet which, when passed over a magnetic receiver of the stimulator 3, results in the stimulator ceasing to provide stimulation signals for contracting the sphincter for control of the neosphincter, in embodiments stimulating a neosphincter or sling.

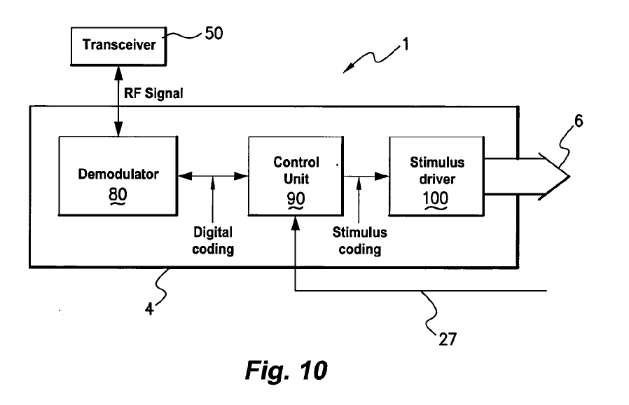

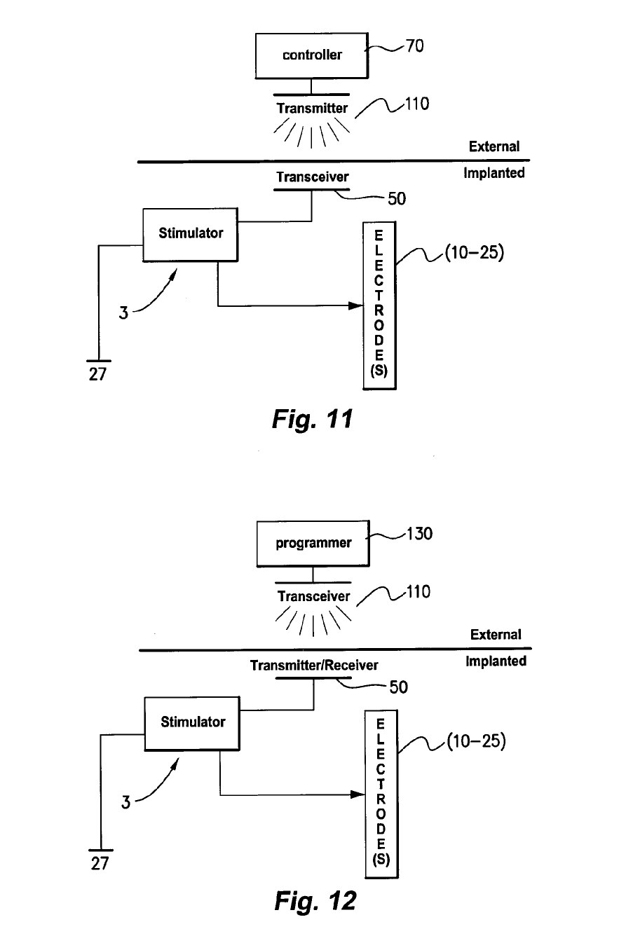

FIGS. 10 through 12 show embodiments of apparatus in accordance with the present invention wherein the stimulator is arranged to receive feedback from a sensor 27, such as a pressure sensor (embodiments of FIG. 5 and FIG. 6) fed back to the control unit 90. Same reference numerals have been used in FIGS. 10 through 12 as were used for the same components in FIGS. 8, 10 and no further description will be given.

Control unit 90 in this case is arranged to receive a signal from one or more sensors 27. In this embodiment the sensor is a pressure sensor which is arranged to detect a change of pressure within the abdominal area, which may be indicative of a “stress” event such as a laugh or a cough. Such events are normally associated with a greater likelihood of leakage, where a person has continence problems.

In response to detection of such a stress event, the control unit 90 controls the stimulus by the stimulus driver 100 to provide a stimulation signal to an appropriate one or more electrodes. For example in the embodiments of FIGS. 5 and 6, the dynamics sling 25 is “pulled up” in response to detection of a stress event stimulators to a neosphincter or sling may be increased.

Other sensors than pressure sensors may be distributed in the abdomen or on the bladder to sense other parameters, e.g. bladder fullness (a stretch sensor mounted on the bladder), and control stimulation of one or more electrodes accordingly. In the case of bladder fullness, when the bladder is full, stimulation of a dynamic sling may pull the sling up further so as to increase the resistance to leakage.

In the above embodiments, power sources for the implantable stimulator will be provided in the form of batteries. These are not shown in the diagrams. The batteries may be replaceable or may be rechargeable via inductive recharging.

As discussed above, the stimulator implant is preferably sealed and encased in a biologically inert material such as a bio-compatible silicone material. Metallic electrodes and leads are preferably of platinum-iridium alloy. The connecting wires are preferably insulated with a silicone coating. The implant is preferably placed between the abdominal muscle and the skin.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An apparatus for treating a condition associated with vaginal prolapse, the apparatus including an implantable stimulator arranged to be implanted within a patient and to provide a signal for stimulation of a contractile tissue sling in the pelvic region, in order to treat the condition.

2. An apparatus in accordance with claim 1, wherein the condition is associated with corrective surgery which has been carried out to treat the prolapse.

3. An apparatus in accordance with claim 1, wherein the implantable stimulator is arranged to provide a signal to contractile tissue which is positioned about the urethra in order to contract the urethra and maintain continence.

4. An apparatus in accordance with claim 3, wherein the contractile tissue is a smooth muscle sphincter.

5. An apparatus in accordance with claim 3, wherein the contractile tissue sling is a smooth muscle sling which partially surrounds the urethra.

6. An apparatus in accordance with claim 3, wherein mesh or some other flexible, biocompatible material is provided to support the sling or sphincter and urethra and/or bladder.

7. An apparatus in accordance with claim 3, wherein the sling or sphincter is cradled by a mesh sling.

8. An apparatus in accordance with claim 7, wherein electrodes are provided on the mesh to stimulate surrounding tissue in the pelvic area.

9. An apparatus in accordance with claim 3, wherein the contractile tissue sling is implanted smooth muscle tissue or skeletal muscle tissue.

10. An apparatus in accordance with claim 4, wherein the smooth muscle sling or sphincter is stimulated to maintain coaptation of the urethra, and a relatively rigid support is provided to contact the sphincter or sling and provide support therefore.

11-12. (canceled)

13. An apparatus in accordance with claim 1, wherein the contractile tissue sling is implanted and arranged to elevate and support the bladder on contraction in order to facilitate urinary continence.

14-25. (canceled)

26. An apparatus in accordance with claim 1, wherein the stimulator is arranged to stimulate an area which has been reinforced by mesh or a sling during surgery to correct the prolapse, in order to promote healing.

27. (canceled)

28. An apparatus in accordance with claim 1, wherein the stimulator is arranged to stimulate the wall of the vagina or adjacent pelvic tissue outside of the vaginal vault in order to facilitate healing and tightening of the wall of the vagina following surgery for prolapse.

29-31. (canceled)

32. An apparatus in accordance claim 26, wherein in the wall of the vagina, pelvic floor muscles or other areas of the pelvic anatomy, the electrodes are discretely placed and/or mounted by mesh which is in place to reinforce the pelvic region.

33. An apparatus in accordance with claim 1, wherein the implantable stimulator is further arranged to deliver one or more signals to other areas of the pelvic anatomy in order to promote healing, stimulate the wall of the vagina, stimulate one or more pelvic floor muscles or other areas of the pelvic anatomy.

34. An apparatus in accordance with claim 1, wherein a sensor or sensors are provided for sensing a stress event.

35-75. (canceled)

76. A method of treating a condition associated with vaginal prolapse, the method comprising the steps of providing electrical stimulation to a contractile tissue sling positioned within the pelvic anatomy to treat conditions associated with vaginal prolapse.

77. A method in accordance with claim 76, wherein the condition is associated with corrective surgery which is being carried out to treat the prolapse.

78. A method in accordance with claim 76, wherein the step of providing electrical stimulation comprises the step of providing a signal to contractile tissue which is positioned about the urethra in order to contract the urethra and maintain continence.

79-80. (canceled)

81. A method in accordance with claim 78, wherein mesh is provided to support the sling and/or sphincter and urethra and/or bladder.

82. A method in accordance with claim 76, wherein the step of providing electrical stimulation comprises providing stimulation to surrounding tissue in the pelvic area via electrodes mounted by the mesh.

83. (canceled)

84. A method in accordance with claim 76, wherein the step of providing stimulation includes the step of applying stimulation to stimulate contractile tissue sling which is arranged to elevate and support the bladder and/or urethra.

85. A method in accordance with claim 84, wherein mesh is provided to further support the bladder.

86. A method in accordance with claim 85, wherein the step of providing electrical stimulation comprises providing stimulation to surrounding pelvic tissue via electrodes mounted by the mesh.

87-91. (canceled)

92. A method in accordance with claim 76, wherein the step of applying stimulation includes applying stimulation to the wall of the vagina in order to facilitate tightening of the vaginal wall following surgery for the correction of prolapse.

93. A method in accordance with claim 76, including the further step of detecting an increase in abdominal pressure brought about by a stress event, and applying stimulation to the contractile tissue in response to the increased pressure.

94-95. (canceled)

96. A method in accordance with claim 76, wherein the step of stimulation includes stimulation of muscles lining the length of the urethra.

97. A method in accordance claim 76, wherein the step of stimulating includes stimulating the various muscles of the pelvic floor.

98-131. (canceled)
